(19) United States
(12) Patent Application Publication
(10) Pub. No.: US 2009/0023156 A1
(43) Pub. Date: Jan. 22, 2009

(54) Methods and reagents for quantifying analytes

(76) Inventors: Karl O. Voss, Foster City, CA (US); Arunashree Bhamidipati, Palo Alto, CA (US)

Correspondence Address: MORGAN, LEWIS & BOCKIUS, LLP., 2 PALO ALTO SQUARE, 3000 EL CAMINO REAL, PALO ALTO, CA, 94306, US

(21) Appl. No.: 11/975,105

(22) Filed: Oct. 16, 2007

Related U.S. Application Data
(60) Provisional application No. 60/961,371, filed on Jul. 20, 2007.

(51) Int. Cl. G01N 33/53

(52) U.S. Cl. 435/7.1

(57) ABSTRACT

The present invention relates to methods and reagents for quantifying analytes. More specifically, the present invention related to methods and reagents for measuring one or more analytes present in a sample.

### RELATED APPLICATIONS

This application claims the benefit of, and priority to, U.S. Provisional Patent Application Ser. No. 60/961,371, filed on Jul. 20, 2007, entitled “Method and Reagents for Quantifying Analytes,” the entire disclosure of which is hereby incorporated by reference.

### FIELD OF INVENTION

The present invention relates generally to methods and reagents for quantifying analytes. More specifically, the present invention related to methods and reagents for measuring one or more analytes present in a sample.

### INTRODUCTION

Techniques for determining the absolute or relative abundance of an analyte in a sample are integral to the physical and biological sciences. Such quantitation is usually performed by comparative measurement to know standards. A good standard is a controlled and well characterized substance that behaves proportionally to the analyte in the measuring application. A standard may be an “internal standard” that is added to the samples for purposes of normalizing differences in the detector from measurement to measurement. A standard may also be a “calibration standard” used to build a standard curve that will be used to determine the concentration of an analyte. Often the standard serves both roles such as in “standards addition” experiments.

Often the analyte to be measured is a specific protein in a complex mixture such as a buffer, cell lysate, or tissue sample. Methods for measuring the total amount of protein in a sample include but are not limited to the Bradford Assay Bradford, M M. Analytical Biochemistry 72:248-254 (1976), the Lowry Assay, Lowry, O H. et al., J. Biol. Chem. 193: 265 (1951), spectroscopic absorbance, and bicinchoninic acid assay (BCA), Smith, P. K., et al. Anal. Biochem. 150:76-85 (1985). Measuring the abundance of a single protein in a complex mixture such as a cell lysate is more generally difficult. If the protein's activity is not directly measurable such as by an enzyme assay, immunological means may be used. Most immunoassays are methods that measure the presence or abundance of a substance in a sample by means of a reaction between an antibody and an antigen. Quantitative immunoassays were first developed over 100 years ago starting with the discovery of the precipitin reaction, Kraus, R and Wiener Klin. Wochenscher, 10: 736 (1897); Kabat E. A. and Mayer. M. M. Experimental Immunochemistry, Charles C. Thomas Press, Springfield, Ill. (1948); Modern immunoassays include but are not limited to, the western blot, Towbin H et al., PNAS, 76:4350-4 (1979); Burnette W. N., Anal Biochem 112: 195-203 (1981), enzyme linked immunosorbent assay (ELISA), Yalow, R. and Berson S. J. Clin. Invest. 39:1157-75 (1960); Engvall E and Perlman P, Immunochemistry 8:871-4 (1971); Van Weemen B K and Schuurs A H, FEBS Letters 15: 232-6 (1971), protein arrays, flow cytometry, and the Firefly nano-capillary immunoassay, O'Neill R. A. et al., PNAS, 103:16153-16158 (2006).

Immunological methodologies rely on the specificity and binding kinetics of an antibody to an antigen. Antibodies are large proteins used by the immune system of animals to protect against foreign objects within the body. The structure of antibodies is well known to those in the art. Antibodies are comprised of several classes. All contain constant regions and variable regions. The variable regions are generally where antibodies bind to their targets. Antibodies are often altered in a large variety of ways including cleavage (as in FAbs), directed mutation (as in humanized antibodies), or labeling.

Antibodies are produced by introducing antigens into an animal and inducing an immune response. Antibodies produced against the antigen can then be purified from the blood serum. This mixture of antibodies is said to be polyclonal because it contains many antibodies each capable of binding to multiple regions (epitopes) of the antigen. When a polyclonal antibody is used as a probe the signal can be quite strong due to multiple binding events. However, background is also likely to be higher because of nonspecific binding (cross reactivity) of members of the antibody population to non-target antigens. In practice, each batch of polyclonal antibodies is different even when it is produced using the same antigen.

In contrast, a monoclonal antibody is produced from an immortalized lymphocyte cell line grown in cell cultures or xenographes. These cell lines produce a single antibody that binds to one epitope. Because monoclonal antibodies bind to a single epitope on the antigen there is usually a one to one ratio of antibody to analyte. The resulting signal is usually less then a polyclonal antibody. However, background from a monoclonal antibody can be much lower than a polyclonal if the epitope is specific to the target analyte and the antibody does not cross react with other components in the complex mixture. Monoclonal antibodies have the further advantage of being pure, easy to characterize, and more consistent from batch to batch. Many other (and more subtle) differences between monoclonal and polyclonal are well known to those in the art.

In both cases the means of producing antibodies to a target begins the same way, by inducing an immune response in an animal. This is often performed by injecting an animal with a purified protein. This causes a broad immune response to the entire protein. It is becoming more common to inject an animal with a polypeptide of the specific region of the protein Hogue-Angeletti, R., Journal of Biomolecular Techniques, 10:2-10 (1999). This can add specificity to the immune response and help lower cross reactivity of the induced antibody to undesired targets.

Antibody/epitope interactions are described by two terms, affinity and avidity. Affinity is the binding strength of a single antibody/epitope interaction. It can be expressed mathematically by the affinity constant Ka (Equation #1):

$$Ka = \frac{[ Ab \cdot Ag ]}{[ Ab ] * [ Ag ]} \qquad Equation \#1$$

, where Ab and Ag are the concentrations of antibody and antigen respectively. Affinity constants generally range from 10⁴ mol⁻¹ to 10¹³ mol⁻¹. The higher the affinity constant, the stronger the binding interaction.

Avidity is the summation of the affinity of multiple antibody/epitope interactions when there are multiple points of contact. For most commercial monoclonal antibodies the affinity and avidity of an antibody is the same because the proteins only bind to each other once. Polyclonal preparations may contain multivalent antibodies that can bind multiple times, making the avidity the more correct term. In the instant invention the term affinity includes avidity as well.

The usefulness of an antibody as a probe often is dependent on the specificity of the antibody for its antigen. The specificity can be described as the affinity of the probe for the analyte divided by the affinity for other targets in the complex mixture. Generally the specificity of every clonal antibody is different.

In other words, two different monoclonal antibodies will bind to the same protein with completely different affinity constants and specificities. Therefore it is difficult to apply the standard curve produced by one antibody to a second antibody. Thus, the variety and complexity of antigen antibody interactions complicates their use in quantitative analysis and so there is a need for improvement.

Immunoassays will frequently make use of multiple antibodies. A sandwich-type ELISA makes use of one antibody to capture an analyte and a second reporter-conjugated antibody to detect and quantitate the analyte. Western blots will use an analyte-specific primary antibody to “tag” the analyte and a reporter-conjugated secondary antibody (that binds specifically to the primary antibody) to perform detection and quantitation. A flow cytometer may make use of several antibodies each targeted to a different antigen and labeled with a different dye to perform a multiplex analysis.

Reporter moieties used in immunoassays include but are not limited to radioactivity, fluorescence, chemiluminescence, surface plasmon resonance, and more. The advantages and disadvantages of performing quantitation with these methods are well known to those in the art, and there is continuing need for improvement.

Immunoassays can make use of reagents that share many of the properties of antibodies. These include but are not limited to phage display, yeast display, ribosome display and many other methods of producing proteins with epitope-specific binding behaviors. Further, antibodies can be altered themselves to change by recombinant techniques and mutagenesis to change and improve or their binding characteristics.

In the biological and physical sciences there are many ways of measurement and commercial instruments that perform them.

The western blot was first developed in 1979 by two groups independently Renart J. et al., PNAS, 76:3116-3120 (1979); Towbin H., et al., PNAS, 76:4350-4354 (1979). Improvements in western blotting techniques since that time have been incremental to non-existent with the process being little changed since then. Commercially improvements have been largely in the electrophoretic apparatuses, sizing standards, detection chemistries, and pre-cast gel formats. Products are sold by a large number of companies including but not limited to, Bio-Rad, GE Healthcare, and GenScript Corp. The vast majority of westerns are performed on samples separated by SDS-polyacrylamide gel electrophoresis (SDS-PAGE).

Nearly all attempts to perform quantitation in western blots suffer from huge variation introduced through out the protocol from the loading of the sample to the measurement of the signal. Quantitation is most commonly performed by normalizing to a protein that is not related to the analyte, such as actin. This serves to correct for experimental preparation and loading differences between lanes; but it can not be used for absolute quantitation. Absolute quantitation is usually performed by loading a standard in lanes adjacent to the sample of interest and relating the signal back to the standard. However, this approach suffers from many of the sample and detection errors discussed earlier and as such there is still a need for improvement.

A western immunoblot can be performed on a sample that was separated by isoelectric focusing, although this is much less common than SDS-PAGE. Isoelectric focusing is a well known method of separating biomolecules that dates back to 1912, Ikeda, K. and Suzuki, S. U.S. Pat. No. 1,015,891; Righetti, P. G. and Drysdale, J. W. Laboratory Techniques in Biochemistry and Molecular Biology: Isoelectric focusing, American Elsevier Publishing Co., Inc. (1976). A molecule in an electric field will migrate towards the pole (cathode or anode) that carries a charge opposite to the net charge carried by the molecule. This net charge depends in part on the pH of the medium in which the molecule is migrating. Solutions can be produced that contain a gradient of different pH values at each end of the electric field. As the molecule migrates through the gradient it will encounter different pH conditions and gain or lose protons becoming more neutral in its net charge. Eventually it may reach a spot where its net charge is zero (i.e., its isoelectric point) and it is thereafter immobile in the electric field. Thus, this electrophoresis procedure separates molecules according to their different isoelectric points.

A diverse number of products are available commercially that practice this method of separation including, Protean IEF Cell and Rotofor Purification System from Bio-Rad (Hercules, Calif.), the Agilent (Santa Clara, Calif.) 3100 Offgel Fractionator, the Ettan™ IPGphor™ 3 and Multiphor II Electrophoresis System from GE Healthcare (Piscataway, N.J.), and the P/ACE MDQ Capillary Electrophoresis System from Beckman Coulter (Fullerton, Calif.). Some of these systems are analytical; some are preparative; some are both. Most are in gels (or gel strips), some are in solution inside capillaries. Isoelectric focusing is the first separation dimension used in 2D gel electrophoresis.

Isoelectric focusing and immunoassays have long been practiced together. In fact Towbin et. al. (1979) performed western blots on samples that had been separated in 2D gel electrophoresis in their seminal paper describing the technique. Quantitative IEF immunoassays were first performed within gels, Stibler, H. et al., Pharmacol. Biochem. Behav 13(suppl 1):47-51, 1980, and later as immunoblots, Xin Y. et al., Alcohol Clin Exp Res 15:814-821 (1991); Hartree, E F., Anal Biochem 48:422-427 (1972); Layne, E. Methods in Enzymology 3:447-455 (1957). When quantitation was performed with standards they were invariably added to a separate lane of the gel either as a pI marker or as a quantitative marker to produce a standard curve. When used to produce a standard curve the data suffered from variability due to small differences in conditions through the protocol from lane to lane. These include differences in loading volumes, diffusion, transfer consistency, probing efficiency, and detection efficiency that exist because the standard is not located in close proximity to the analyte.

There has been reported on standards in quantitative immuno assays, Bean P. et al., Carbohydrate-Deficient Transferring Evaluation in Dry Blood Spots, Alcoholism: Clinical And Experimental Research, Vol. 20, No. 1 (1996), capillary immunoassays, and capillary electrophoresis, Bean P., A New Frontier for Capillary Electrophoresis: Detection and Confirmation of Alcohol Abuse, American Biotechnology Laboratory. Vol. 24, No. 6 (2006). However, none of these provides a method encompassing all aspects of the current invention. For example, when the use of standards is mentioned they are not used as standards added to the sample, e.g. U.S. Pat. No. 5,622,871, but rather as experiments performed in parallel. These reported works failed to take advantage of having the internal standard and the analyte manipulated and detected identically. Thus, new developments and improvements are needed and there is a continuing need for new methods and reagents for quantifying analytes.

### SUMMARY OF THE INVENTION

In some embodiments, the present invention is directed to a method of measuring one or more analytes in a sample comprising the steps of: adding a known quantity of a standard to the sample; resolving the standard and the analyte by electrophoresis; detecting the standard and the one or more analytes with one or more detection molecules, reagents, or agents such as one or more antibodies; and comparing the signal of the standard to the signal of the one or more analytes. In some embodiments the standard shares a binding site for the detection molecule with the analyte.

In some embodiments the analyte and the standard are polypeptides or proteins. In still other embodiments the standard and the analyte share an epitope. In some embodiments, a method of measuring one or more analytes in a sample is provided comprising: adding a known quantity of a standard to the sample; loading the sample into a microfluidic device; separating the standard and the analyte by electrophoresis; immobilizing the standard and the analyte; detecting the standard and analyte with at least one antibody; and comparing the signal of the standard to the signal of the analyte to determine the quantity of analyte in the sample. In another aspect of the present invention kits for performing the methods described herein, and for systems for quantifying analytes are provided. In one embodiment, the kit comprises materials for quantifying analytes including one or more antibodies, one or more internal standards. Additionally, indications of the concentration of the one or more internal standards may be included. In some embodiments, the kit further includes one or more standards comprised of a molecule that shares an epitope with an analyte of interest. Additional materials can include, but are not limited to, fluid paths, such as capillaries and microfluidic devices. In addition, buffers, polymeric or polymerizable materials, blocking solutions, and washing solutions can be provided. In some embodiments, the kit can further comprise reagents for the activation of a reactive moiety. These other components can be provided separately from each other, or mixed together in dry or liquid form.

### BRIEF DESCRIPTION OF THE FIGURES

The skilled artisan will understand that the drawings, described below, are for illustration purposes only. The drawings are not intended to limit the scope of the present teachings in any way.

FIG. 1 depicts one embodiment of a system according to the present invention in which a standard is first added to a sample to be analyzed;

FIG. 2 is a schematic diagram illustrating one embodiment of the method of the present invention carried out in a nano-capillary immunoassay;

FIG. 3 shows data extracted from 12 replicate samples prepared according to experiments in Example 1 described below, according to some embodiments of the present invention;

FIG. 4 presents a TIFF image from a CCD camera of the 12 replicate samples prepared according to experiments in Example 3 described below, according to some embodiments of the present invention;

FIG. 5 shows data extracted from a standards addition experiment that illustrates how the invention can be used to determine the amount of one or more analytes in an unknown sample;

FIG. 6 shows the data from FIG. 5 plotted on a graph as peak area vs. concentration of the standard; and

FIG. 7 depicts a standard curve created by analyzing the peak area data extracted from FIG. 5 according to embodiments of the present invention.

### DETAILED DESCRIPTION OF THE INVENTION

It is to be understood that both the foregoing general description and the following description are exemplary and explanatory only and are not restrictive of the methods and devices described herein. In this application, the use of the singular includes the plural unless specifically stated otherwise. Also, the use of “or” means “and/or” unless state otherwise. Similarly, “comprise,” “comprises,” “comprising,” “include,” “includes,” “including” “haves” and “having” are not intended to be limiting.

The present invention is generally directed to biological and chemical applications requiring internal and/or calibration standards used to determine measurement of an analyte or molecule of interest. Applications include but are not limited to proteins separated by electrophoresis and immobilized within capillaries.

In one aspect, the present invention is directed to the use of one or more internal standards for measuring or quantifying analytes. Generally, a known quantity of standard is added to a sample comprising one or more analytes, and both the standard and the sample are separated by capillary electrophoresis. Then the standard and the analyte(s) are detected with one or more detection molecules or reagents, such as with an antibody. The signal of the standard and the signal of the analyte(s) are compared to measure the analyte(s) in the sample.

In one aspect, the present invention provides an internal standard. By “standard” or “internal standard” herein is meant a well characterized substance of known amount that is added to an unknown analyte for comparative purposes. In some embodiments, an internal standard is a purified form of the analyte itself, although it is generally preferred that the standard be distinguishable from the analyte in some way. This can be performed by a variety of ways, such as by making trivial changes that do not alter the relevant properties of the standard. Preferably an internal standard is different from the analyte but behaves in a way similar to the analyte, enabling relevant comparative measurements.

In some embodiments the standard of the invention is comprised of a purified and well characterized form of the analyte. Any method of obtaining a pure form of the analyte is compatible with the invention, including but not limited to purification from nature, purification from organisms grown in the laboratory, by chemical synthesis and the like.

In some embodiments the standard of the invention has been altered in some way that distinguishes it from the unknown when detected in the detection step or system. The distinguishing characteristic can be any change that is compatible with the invention, including but not limited to dye labeling, radiolabeling, or modifying the mobility of the standard during the electrophoretic separation so that it is separated from the analyte. For example, a standard can contain a modification of the analyte that changes the charge, or mass, or length of the standard relative to the analyte of interest. Modifications include but are not limited to a deletion, fusion, or any chemical modification.

In some embodiments the internal standard is comprised of a peptide or polypeptide that shares at least one epitope with the analyte of interest. As described herein, antibodies are frequently made by injecting a polypeptide containing a portion of the analyte protein sequence rather than the entire sequence. Antibodies raised to these polypeptides are then selected for their ability to bind to the analyte as well as the polypeptide. These polypeptides make excellent internal standards since they share an epitope with the analyte of interest. There are multiple means of synthesizing polypeptides as is well known to those practiced in the art. They can be synthesized on commercially available instruments, or in living organisms using recombinant DNA technology known in the art.

In another embodiment the standard is a molecule that shares an epitope with the analyte. These molecules may exist naturally in the form of natural homologues or they can be created by recombinant techniques well known to molecular biologists, or they can mimetics created by synthetic chemistry. In some embodiments the standard is a fusion protein, also know as a chimeric protein. Such a protein is created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics.

In some embodiments the standard is a protein that binds immunoglobulins. Examples of this embodiment include but are not limited to Protein A of the bacterium Staphylococci and protein G from Streptococci. These particular examples are both proteins that happen to bind to a region found on immunoglobulins called the constant region rather than the variable region of the antibody. Most antibodies raised to analytes will contain this region and so will bind both to the analyte of interest and to these proteins in the antibody binding step. Standards can be liquid or solid form as long as the amount of the standard is known, constant, or determinable after the assay is carried out.

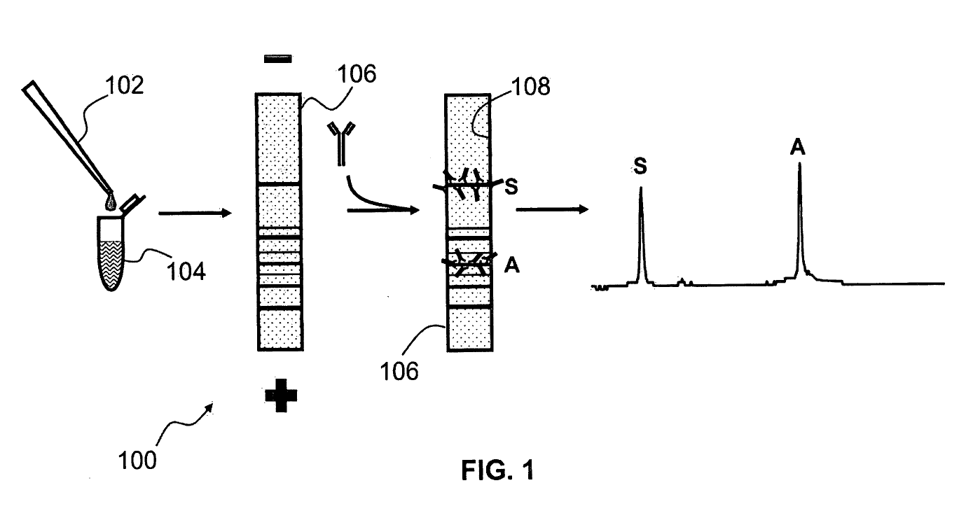

In another aspect, the present invention provides immunoassays for detecting analytes. FIG. 1 depicts one embodiment of a system 100 according to the present invention in which a standard 102 is first added to a sample 104 to be analyzed. The sample 104 is then subjected to an electrophoretic separation in a capillary 106 in which the standard (S) and the analyte (A) are separated from each other. Then a potion or the entire sample is immobilized to a wall 108 of the capillary 106. The sample and the standard are then probed simultaneously with a single detection molecule, reagent or agent that binds to both the standard (S) and the analyte (A) of interest. The detection reagent is then used to create a signal that can be detected and graphed as signal vs. length of the capillary. The signal from the standard (S) can then be used to interpret the signal from the analyte (A).

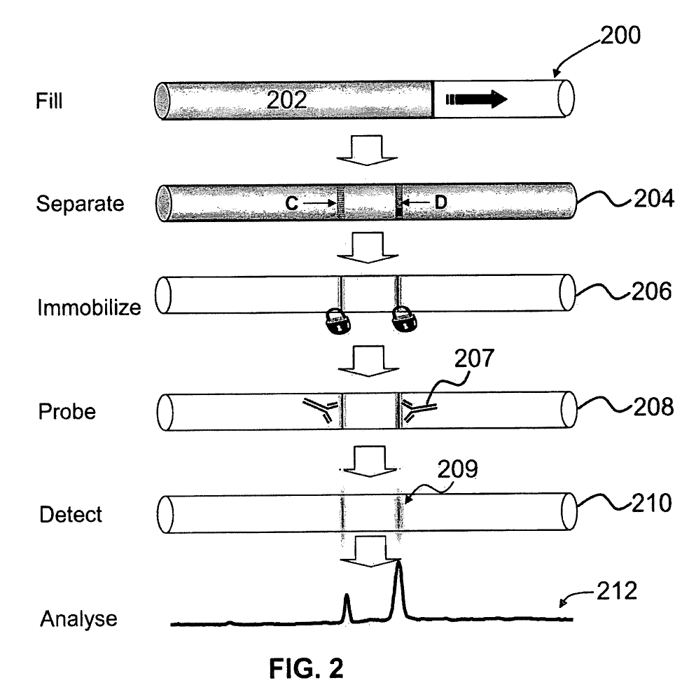

In one aspect, the present invention provides methods that can be used with capillary electrophoresis systems, including but not limited to, nano-capillary immunoassay systems, such as the Firefly™ immunoassay system available from Cell Biosceiences Inc. The Firefly nano-capillary assay automates the fundamental steps of the classical western blotting process at nanoliter volumes with extremely high sensitivity. All steps of the process occur in the nano-volume capillary chambers and are described in O'Neill et al, PNAS, 103: 16153-16158 (2006), herein incorporated by reference by its entirety. The nano-volume capillaries were prepared as described in U.S. patent application Ser. No. 11/654,143, which is herein incorporated by reference in its entirety. FIG. 2 is a schematic diagram illustrating one embodiment of the method of the present invention carried out in a nano-capillary immunoassay, such as but not limited to the Firefly immunoassay. Briefly, the capillaries 200 are loaded with a separation medium and the sample 202. The contents are resolved by capillary electrophoresis shown at 204. Resolved proteins are then immobilized to the capillary wall as shown at 206. The immobilized proteins are then probed with an analyte-specific antibody, and a HRP-conjugated secondary antibody in a manner similar to a western blot as shown at 208. Because the protein-antibody complexes are immobilized in the capillary, detection molecules or reagents can be flowed through the capillary as shown at 210. Light generated from where the antibodies bound is imaged onto a CCD camera. Data is extracted and plotted as signal intensity vs. capillary length with the analyte showing up as a peak as shown at 212. Fluorescent standards are used to align the data from capillary to capillary. Also within the scope of the present invention are a variety of variations on the methods described above.

By “capillary” herein is meant a bore or channel through which a liquid or dissolved molecule can flow. A capillary can be comprised of any convenient material, such as glass, plastic, silicon, fused silica, gel, or the like. In some embodiments, the method employs a plurality of capillaries. A plurality of capillaries enables multiple samples to be analyzed simultaneously

The capillary can vary as to dimensions, width, depth and cross-section, as well as shape, being rounded, trapezoidal, rectangular, etc., for example. The capillary can be straight, rounded, serpentine, or the like. Suitable sizes include, but are not limited to, capillaries having internal diameters of about 5 to about 1000 μm, although more typically capillaries having internal diameters of about 10 to about 400 μm can be utilized.

Embodiments of the invention include separation of the analytes by any physical characteristic, including but not limited to their size, pI, or charge to mass ratio, hydrophobicity, etc. The analytes can be immobilized by any methods compatible to the present invention, including but not limited to chemical, photochemical, and heat treatment. Detection of the analytes can be performed by any methods compatible with the invention, including but not limited to chemiluminescence, staining, autoradiography, fluorescence, and the like.

The nanocapillary immunoassay is an ideal (though not the only) application for the invention because there are discrete measurements taken in each capillary that need to be compared from capillary to capillary, run to run, instrument to instrument, day to day. Internal quantitative standards enable the normalization of differences in the data due to changes in the sample or the detector.

Sample preparation is preformed as follows. By “sample” herein is meant a composition that contains the analyte or analytes to be detected. The sample can be heterogeneous, containing a variety of components, i.e. different proteins. Alternatively, the sample can be homogenous, containing one component. The sample can be naturally occurring, a biological material, or man-made material. The material can be in a native or denatured form. The sample can be a single cell or a plurality of cells, a blood sample, a tissue sample, a skin sample, a urine sample, a water sample, or a soil sample. In some embodiments, the sample comprises the contents of a single cell, or the contents of a plurality of cells. The sample can be from a living organism, such as a eukaryote, prokaryote, mammal, human, yeast, or bacterium, or the sample can be from a virus.

The analyte to be detected can be any analyte of interest selected by the user. In some embodiments an analyte is any substance that elicits an immune response either directly or as a hapten-conjugate. While antigens are usually polypeptides and proteins, they may also be polysaccharides, lipids, nucleic acids, or other class of organic molecule. The analyte can comprise any organic or inorganic molecule capable of being detected. For example, analytes that can be detected include proteins, oligopeptides and peptides, derivatives and analogs, including proteins containing non-naturally occurring amino acids and amino acid analogs. Other examples of analytes that can be detected include carbohydrates, polysaccharides, glycoproteins, viruses, metabolites, cofactors, nucleotides, polynucleotides, transition state analogs, inhibitors, drugs, nutrients, electrolytes, hormones, growth factors and other biomolecules as well as non-biomolecules, as well as fragments and combinations thereof.

In one aspect, the present invention provides method of using of an internal standard for quantitation. An ideal and precise measuring device reproducibly produces a readout that changes in exact proportion to the amount of analyte in the sample. A less precise device or a changing sample will require an internal standard to compensate for these “matrix effects” in order for an accurate measurement to be made. In other cases an internal standard is added in different (but defined amounts) to produce a signal that varies with amount. This can be made into a graph plotting signal on the Y axis vs. amount of the standard on the X axis. The signal of the unknown analyte can then be matched to the graph to determine the amount of the unknown in “standard units”.

In some embodiments, the use of an internal standard includes adding a known amount of a standard to a sample, then measuring the standard and the analyte concurrently. Variations in the measurement of the standard are then used to adjust the reading of the analyte to compensate for changes that occurred due to changes in the detector or treatment of the samples from measurement to measurement.

In some embodiments, different known amounts of the standard are added to the sample in a process known as standards addition. The amount of standard added typically spans the range of amounts expected to be observed for the analyte or is titrated down to a zero point. A graph, called a standard curve, is produced by plotting the observed measurement versus the amount of standard. In some applications the signal from the analyte can be compared to the signal of the standard to determine the amount of analyte in the sample. In other applications the Y intercept of the curve is used to determine the analyte concentration.

Measurements are often repeated many times on both the standards and analytes so that the precision of the measurement can be determined statistically.

In some embodiments the standard is combined with and mixed with the sample. The standard can be added to the sample, by any methods compatible with the invention, including but not limited to, pipetting, pouring, dripping, spooning, hydrodynamic flow, and the like. Introduction of the standards could also be via electrokinetic methods such as electro injection, electroosmotic flow, or electrophoresis. There are a wide variety of methods used in microfluidics that are also compatible with the invention that move and mix reagents. What is important is that the amount of standard be added in a known amount, a consistent amount, or an amount determinable after the fact.

As will be appreciated by those skilled in the art the standard can be added to the sample at any point and by any methods as long as it is compatible with the invention. What is important is that the amount of the standard in the sample be known, constant, or determinable after the fact. The timing for addition of the internal standard is generally important for data interpretation. Generally, the standard can be added at any processing step prior to addition to the capillary depending on the goal of the investigator.

In some embodiments the standard is added to the sample immediately prior to loading into the capillary for electrophoresis. Addition of the standard at this step controls for changes due to manipulations during separation, immobilization, probing, and detection. In other embodiments, the standard is added to the sample at the earliest stages of sample preparation to control for changes in the sample during the selected manipulations as well as the addition to the capillary through detection. Examples include but are not limited to adding the standard before a lysing step, denaturation step, heating step, purification step, precipitation step, immunoprecipitation step, column chromatography step, or centrifugation, and the like.

In some embodiments standards are added at multiple steps in parallel to study the effect of an intermediate step. For example a blood sample may be divided into two samples. A standard may be added to one just prior to lysing. The samples can then be processed in parallel. Standard can be added to the other sample just prior to addition to the capillary. Analysis of the first sample may indicate the total variation introduced into the process. Analysis of the second sample will examine the variation introduced part way through the process. Subtraction of the partial from the total may inform the experimenter the variation that was introduced at the beginning of the manipulations. By means such as these, standards addition can be used to determine the sources of variation some or all of the stages of sample preparation and analysis.

The sample can be loaded into the capillary by any methods compatible with the invention. In some embodiments the methods of introducing the sample into the capillary is by hydrodynamic forces such as but not limited to all forms of pressure, capillarity (surface tension), gravity, sonics, etc. In some embodiments the capillary is used as a pipette tip and is dipped into the sample. In another embodiment the sample is introduced by electrokenetic methods such as electrokenetic injection, eletrophoresis, or electro osmotic flow. Those skilled in the art will be familiar with the large range of methods and prior art used by the microfluidics community for manipulating samples and moving them from one location to another using combinations of hydrodynamic, thermal, electrical, and magnetic forces. A sample may be introduced to one end of a capillary it may be used to completely fill a capillary depending on the type of separation that is to be performed.

The methods generally comprise resolving the one or more analytes, contained in a sample. Methods of separating a mixture into two or more components are well know to those of ordinary skill in the art, and may include, but are not limited to, various kinds of electrophoresis.

In some embodiments the sample and standard are subjected to an electrophoretic separation. By “electrophoresis” herein is meant the movement of suspended or dissolved molecules through a fluid or gel under the action of an electromotive force applied to electrodes in contact with a fluid. In some embodiments the fluid or gel contains one or more buffers. In some embodiments the buffers are carrier ampholytes suitable for isoelectric focusing. In some embodiments, the fluid path comprises a gel. In some embodiments, the gel is capable of separating the components of the sample based on size, length, or molecular weight. A wide variety of such gels are known in the art, a non-limiting example includes a polyacrylamide gel.

In some embodiments, resolving one or more analytes comprises isoelectric focusing (IEF) of a sample. In an electric field, a molecule will migrate towards the pole (cathode or anode) that carries a charge opposite to the net charge carried by the molecule. This net charge depends in part on the pH of the medium in which the molecule is migrating. One common electrophoretic procedure is to establish solutions having different pH values at each end of an electric field, with a gradient range of pH in between. At a certain pH, the isoelectric point of a molecule is obtained and the molecule carries no net charge. As the molecule crosses the pH gradient, it reaches a spot where its net charge is zero (i.e., its isoelectric point) and it is thereafter immobile in the electric field. Thus, this electrophoresis procedure separates molecules according to their different isoelectric points.

In some embodiments, for example when resolving is performed by isoelectric focusing, an ampholyte reagent can be loaded into the fluid path. An ampholyte reagent is a mixture of molecules having a range of different isoelectric points. Typical ampholyte reagents are Pharmalyte™ and Ampholine™ available from Amersham Biosciences of Buckinghamshire, England. Ampholytes can be supplied at either end of the fluid path, or both, by pumping, capillary action, gravity flow, electroendosmotic pumping, or electrophoresis, or by gravity siphon that can extend continuously through the fluid path.

In some embodiments, resolving one or more analytes comprises electrophoresis of a sample in a polymeric gel. Electrophoresis in a polymeric gel, such as a polyacrylamide gel or an agarose gel separates molecules on the basis of the molecule's size. A polymeric gel provides a porous passageway through which the molecules can travel. Polymeric gels permit the separation of molecules by molecular size because larger molecules will travel more slowly through the gel than smaller molecules.

In some embodiments, resolving one or more analytes comprises micellar electrokinetic chromatography (MEKC) of a sample. In micellar electrokinetic chromatography, ionic surfactants are added to the sample to form micelles. Micelles have a structure in which the hydrophobic moieties of the surfactant are in the interior and the charged moieties are on the exterior. The separation of analyte molecules is based on the interaction of these solutes with the micelles. The stronger the interaction, the longer the solutes migrate with the micelle. The selectivity of MEKC can be controlled by the choice of surfactant and also by the addition of modifiers to the sample. Micellar electrokinetic chromatography allows the separation of neutral molecules as well as charged molecules.

Once the analyte and standard are separated, the detection step is carried out. In a preferred embodiment a detection molecule or reagent is an antibody capable of binding to or interacting with the standard and the analyte to be detected. Contacting the detection agent with the analyte or analytes of interest can be by any method known in the art, so long as it is compatible with the methods and devices described herein. Examples for conveying detection agents through the capillary include, but are not limited to, hydrodynamic flow, electroendosmotic flow, or electrophoresis.

Antibodies used for detection of the standard and the analyte may be labeled by any means compatible with the invention such as but not limited to fluorescent dyes, optical dyes, chemiluminescent reagents, radioactivity, particles, magnetic particles, etc. In some embodiments, the detection agents comprise one or more label moiety(ies). In embodiments employing two or more label moieties, each label moiety can be the same, or some, or all, of the label moieties may differ.

In some embodiments, the detection agents comprise chemiluminescent labeled antibodies. The chemiluminescent label can comprise any entity that provides a light signal and that can be used in accordance with the methods and devices described herein. A wide variety of such chemiluminescent labels are known in the art. See, e.g., U.S. Pat. Nos. 6,689,576, 6,395,503, 6,087,188, 6,287,767, 6,165,800, and 6,126,870 the disclosures of which all are incorporated herein by references. Suitable labels include enzymes capable of reacting with a chemiluminescent substrate in such a way that photon emission by chemiluminescence is induced. Such enzymes induce chemiluminescence in other molecules through enzymatic activity. Such enzymes may include peroxidase, beta-galactosidase, phosphatase, or others for which a chemiluminescent substrate is available. In some embodiments, the chemiluminescent label can be selected from any of a variety of classes of luminal label, an isoluminol label, etc.

In some embodiments, the detection agents comprise chemiluminescent substrates. Chemiluminescent substrates are well known in the art, such as Galacton substrate available from Applied Biosystems of Foster City, Calif., or SuperSignal West Femto Maximum Sensitivity substrate available from Pierce Biotechnology, Inc. of Rockford, Ill. or other suitable substrates.

Likewise, the label moiety can comprise a bioluminescent compound. Bioluminescence is a type of chemiluminescence found in biological systems in which a catalytic protein increases the efficiency of the chemiluminescent reaction. The presence of a bioluminescent compound is determined by detecting the presence of luminescence. Suitable bioluminescent compounds include, but are not limited to luciferin, luciferase and aequorin.

In some embodiments, the detection agents comprise fluorescent dye labeled antibodies. The fluorescent dye can comprise any entity that provides a fluorescent signal and that can be used in accordance with the methods and devices described herein. Typically, the fluorescent dye comprises a resonance-delocalized system or aromatic ring system that absorbs light at a first wavelength and emits fluorescent light at a second wavelength in response to the absorption event. A wide variety of such fluorescent dye molecules are known in the art. For example, fluorescent dyes can be selected from any of a variety of classes of fluorescent compounds, non-limiting examples include xanthenes, rhodamines, fluoresceins, cyanines, phthalocyanines, squaraines, bodipy dyes, coumarins, oxazines, and carbopyronines. In some embodiments, for example, where detection agents contain fluorophores, such as fluorescent dyes, their fluorescence is detected by exciting them with an appropriate light source, and monitoring their fluorescence by a detector sensitive to there characteristic fluorescence emission wavelength.

In some embodiments, using two or more different detection agents, which bind to or interact with different analytes, different types of analytes and standards can be detected simultaneously. In some embodiments, two or more different detection agents, which bind to or interact with the one analyte and standard, can be detected simultaneously. In some embodiments, using two or more different detection agents, one detection agent, for example a 1° antibody, can bind to or interact with a standard and one or more analytes to form a complex, and second detection agent, for example a 2° antibody, can be used to bind to or interact with the complex.

Analyte and standard detection can be carried out by any method known in the art, so long as it is compatible with the methods and devices described herein. Detection can be performed by monitoring a signal using conventional methods and instruments, non-limiting examples include, a photodetector, an array of photodetectors, a charged coupled device (CCD) array, and the like. For example, a signal can be a continuously monitored, in real time, to allow the user to rapidly determine whether an analyte is present in the sample, and optionally, the amount or activity of the analyte. In some embodiments, the signal can be measured from at least two different time points. In some embodiments, the signal can be monitored continuously or at several selected time points. Alternatively, the signal can be measured in an end-point embodiment in which a signal is measured after a certain amount of time, and the signal is compared against a control signal (sample without analyte), threshold signal, or standard curve.

The amount of the signal generated is not critical and can vary over a broad range. The only requirement is that the signal be measurable by the detection system being used. In some embodiments, a signal can be at least 2-fold greater than the background. In some embodiments, a signal between 2 to 10-fold greater than the background can be generated. In some embodiments, a signal can be more than 10-fold greater than the background.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

### EXAMPLES

### Example 1

The Internal Standard Normalizing for Capillary to Capillary Variation.

This example lays out detailed specific protocol for practicing one embodiment of the present invention. The cell signaling protein ERK2 is analyzed in a cell lysate, specifically HELA cells. The standard used in this example is a synthesized peptide known to bind to the antibody used to detect ERK. Twelve replicates of the sample containing the internal standard are processed simultaneously using an automated instrument. The lysate and analytes are separated by isoelectric focusing. Detection is by first probing the immobilized proteins with a primary antibody to the target(s) followed by probing the contents with a HRP-conjugated secondary antibody to the primary antibody. Detection is performed by flowing chemiluminescent reagents through the capillary and detecting signal using a CCD camera. The camera produces a 12 bit TIFF image from which the signal can be extracted and analyzed.

Cell lysate preparation. Hela cells were grown in the presence of DMEM (VWR cat 45000-304) medium and lysed before they reached the stationary phase. Medium was aspirated and the plated cells were washed twice with cold HNG buffer containing 20 mM Hepes (pH 7.5), 25 mM NaCl, 10% glycerol and 0.1% protease inhibitor cocktail (cat. no. 539134; Calbiochem). Cells were incubated on ice in cold hypotonic lysis buffer containing HNG and 0.1% Triton (HNTG) for 10 min. Cells were scraped off and transferred into a microcentrifuge tube and incubated in the cold for 1 hr. Lysate was clarified by centrifugation twice. The protein concentration was determined by a bicinchoninate assay (cat. no. 23225; Pierce).

Synthesis of Erk1 Peptide Standard. The ERK immunogen peptide sequence was obtained from the manufacturer (Millipore Corp, Billerica, Mass.) of the cognate antibody. The peptide, sequence PFTFDMELDDLPKERLKELIFQETARFQPGAPEAP (SEQ ID NO: 1), was synthesized on a 0.1 M scale using FMOC chemistry on an Applied Biosystems 433 Peptide Synthesizer using standard protocols, leaving an FMOC group on the terminal proline residue. This protected peptide on resin was then treated in two different ways.

One portion was transferred to glassware, and the terminal FMOC was removed by standard treatment with piperidine. Cleavage and deprotection was followed by preparative reverse-phase HPLC purification to yield 1.0 mg of peptide. Purity was determined to by greater than 90% by HPLC.

Another portion of the protected peptide on resin was transferred to glassware, and the terminal FMOC was then removed by standard treatment with piperidine, followed by treatment with 5-TAMRA-SE under basic conditions (DIEA). After standard deprotection and cleavage, the labeled peptide was purified by preparative reverse-phase HPLC to give 0.3 mg of reddish solid, which was determined to be about 90% pure by HPLC, monitoring at 556 nm. In this example the exact amount of standard added to the sample is not as important as that the, same amount be precisely added to all tubes.

Sample Preparation for IEF. Hela lysate prepared as described above was diluted in HNTG buffer to a concentration of 0.06 mg/ml of total protein and mixed with an equal volume of an IEF buffer solution containing 20% sorbitol, 0.1 M NDSB-256 (cat. no. D465250, Toronto Research Chemicals), 2 μM of pI 4.92 and pI 7.01 fluorescent peptide standards for IEF (Cellbiosciences, Inc.), 10% pI 2-11 ampholytes (cat. no. 764032, Bioworld). The peptide standard was added to the sample to achieve a final concentration of 0.05 nM.

IEF and immobilization. The sample prepared as described above was used in the nanocapillary immunoassay as (described in O'Neill et al.). Replicates were run 12 at a time on 2 different instruments. Proteins were focused at a constant power of 1200 μW/capillary for 2000 second and immobilized by 60 second irradiation with UV light.

Washing and Probing. After UV immobilization, capillaries were washed with a TBST solution containing 10 mM Tris-HCl at pH 7.5, 150 mM NaCl, 0.05% Tween20. Immobilized proteins were then incubated for 1 hr with anti-Erk1/2 antibodies (cat. no. 06-182, Upstate) diluted to 1:300 in TBST. This was followed by another TBST wash to remove non-specifically bound antibodies. Immobilized proteins were then incubated for 10 minutes with HRP conjugated goat anti-rabbit secondary antibodies (cat. no. 81-6120, Zymed) diluted 1:500 in TBST. Capillaries were washed with TBST and chemiluminescence detection was performed using West Femto Stable Peroxide buffer and Luminol/Enhancer solution (cat. no. 1859023, 1859022, Pierce). Images of the capillaries are taken using a CCD camera (Princeton Instruments) for 120 seconds. Images are stored in a 16 bit TIFF file format.

Data analysis software (Cell BioSciences, Palo Alto, Calif.) was used to extract the data from the CCD images within a cycle of 12 capillaries. The observed peak heights of Erk1 protein (G) and the peptide standard (E) were exported to Excel (Microsoft, Redmond Wash.) for further analysis. The data are summarized in Table 1. Using peak area to perform this analysis gives similar results.

TABLE 1

| Capillary | ERK2obs | STDobs | Factor | ERKnorm |
|---|---|---|---|---|
| 1 | 297 | 151 | 0.12 | 2551 |
| 2 | 907 | 551 | 0.42 | 2141 |
| 3 | 1030 | 751 | 0.58 | 1780 |
| 4 | 1220 | 976 | 0.75 | 1620 |
| 5 | 1180 | 1010 | 0.78 | 1520 |
| 6 | 1960 | 1300 | 1.00 | 1960 |
| 7 | 1530 | 1210 | 0.93 | 1650 |
| 8 | 2010 | 1270 | 0.98 | 2050 |
| 9 | 1740 | 1130 | 0.87 | 2000 |
| 10 | 1920 | 1170 | 0.90 | 2130 |
| 11 | 1720 | 1190 | 0.91 | 1880 |
| 12 | 362 | 257 | 0.20 | 1832 |
| AVE | 1323.00 | | | 1926.17 |
| STDEV | 593.50 | | | 280.81 |
| % CV | 45% | | | 15% |

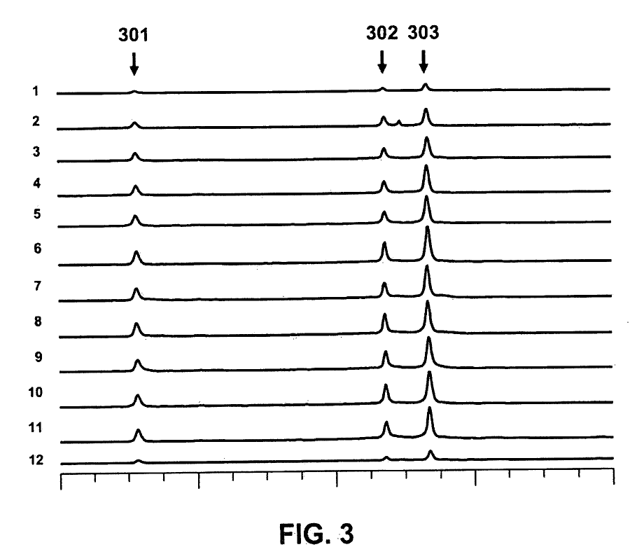

The amount of standard added to each capillary is constant; therefore the amount of standard can be used to normalize the amounts of ERK1 relative to each other. The normalized signal of the analyte ERK is determined by multiplying the observed ERK signal by a conversion factor such as Signal_{STDobs}/Signal_{STDmax} or Signal_{STDobs}/Signal_{STDave} or Signal_{STDobs}/Signal_{STDmin}. For purposes of this example we will use Signal_{STDobs}/Signal_{STDmax}. to calculate a normalization factor. This method will result in the normalized signals of the analyte being generally larger than the observed signals. Capillary #6 had the highest signal so its factor is 1.0. The observed signals are then divided by the normalization factor to give the corrected signal, ERKₙₒᵣₘ. Note that the variation in signal was greatly reduced in this example from a CV of 45% to a CV of 15%. FIG. 3 shows the data extracted from the 12 replicate samples according to experiments described herein in Example 1. These samples were separated by isoelectric focusing. Although the samples all contain equal concentrations of cell lysate and standard, a problem with either the sample, protocol, of detector has introduced variability in the signal between capillaries. However the data shows that all the peak signals rise and fall in unison. The peak produced by the standard (301) can be used to correct the signal in 2 analytes (302 and 303). Thus, the invention greatly reduces variability between capillaries.

### Example 2

The Standard Can Correct for Variation From Day to Day and From Detector to Detector.

This example is much like the first example except that instead of a single run of 12 capillaries being processed simultaneously, several 12 capillary cycles were performed across different days using two different instruments.

The signal from ERK1 was as follows in Table 2:

TABLE 2

| | Day 1 | | | Day 2 | | |
|---|---|---|---|---|---|---|
| | Run 1 | Run 2 | Run 3 | Run 1 | Run 2 | Run 3 |
| | 297 | 464 | 1060 | ND | 509 | 61 |
| | 907 | 1430 | ND | 687 | ND | 649 |
| | 1030 | 1340 | 1320 | 547 | 205 | 289 |
| | 1220 | 1740 | 1280 | 543 | 534 | ND |
| | 1180 | 1800 | 1540 | 532 | 1130 | 512 |
| | 1960 | 1910 | 1500 | 508 | 477 | 455 |
| | 1530 | 1700 | 1350 | 1000 | 749 | 404 |
| | 2010 | 1630 | 1470 | 670 | 583 | 575 |
| | 1740 | 1810 | 1480 | 488 | 498 | 455 |
| | 1920 | 1490 | 1550 | 566 | 486 | 353 |
| | 1720 | 1470 | 1500 | 496 | 535 | 326 |
| | 362 | 329 | 1470 | 484 | 509 | 341 |
| Average | 966 | | | | | |
| Std Dev | 560 | | | | | |
| CV | 58% | | | | | |

The data for the quantitation standard was as follows in Table 3:

TABLE 3

| Observed Standard Signal | | | | | | Normalization Table | | | | | |
|---|---|---|---|---|---|---|---|---|---|---|---|
| Day 1 | | | Day 2 | | | Day 1 | | | Day 2 | | |
| Run 1 | Run 2 | Run 3 | Run 1 | Run 2 | Run 3 | Run 1 | Run 2 | Run 3 | Run 1 | Run 2 | Run 3 |
| 151 | 177 | 690 | 0 | 223 | 0 | 0.12 | 0.14 | 0.53 | — | 0.17 | — |
| 551 | 854 | 0 | 474 | 0 | 318 | 0.42 | 0.66 | — | 0.36 | — | 0.24 |
| 751 | 962 | 775 | 351 | 420 | 237 | 0.58 | 0.74 | 0.60 | 0.27 | 0.32 | 0.18 |
| 976 | 1050 | 854 | 390 | 477 | 0 | 0.75 | 0.81 | 0.66 | 0.30 | 0.37 | — |
| 1010 | 991 | 1100 | 258 | 635 | 329 | 0.78 | 0.76 | 0.85 | 0.20 | 0.49 | 0.25 |
| 1300 | 1040 | 1050 | 376 | 387 | 287 | 1.00 | 0.80 | 0.81 | 0.29 | 0.30 | 0.22 |
| 1210 | 1020 | 992 | 532 | 387 | 466 | 0.93 | 0.78 | 0.76 | 0.41 | 0.30 | 0.36 |
| 1270 | 1220 | 1060 | 488 | 338 | 439 | 0.98 | 0.94 | 0.82 | 0.38 | 0.26 | 0.34 |
| 1130 | 1170 | 915 | 296 | 286 | 262 | 0.87 | 0.90 | 0.70 | 0.23 | 0.22 | 0.20 |
| 1170 | 1110 | 900 | 315 | 364 | 241 | 0.90 | 0.85 | 0.69 | 0.24 | 0.28 | 0.19 |
| 1190 | 977 | 997 | 347 | 306 | 292 | 0.92 | 0.75 | 0.77 | 0.27 | 0.24 | 0.22 |
| 257 | 141 | 889 | 253 | 515 | 203 | 0.20 | 0.11 | 0.68 | 0.19 | 0.40 | 0.16 |

Normalizing the ERK1 data by dividing the ERK observed by the normalization factor one obtains the following result as shown in Table 4:

TABLE 4

| | Day 1 | | | Day 2 | | |
|---|---|---|---|---|---|---|
| | Run 1 | Run 2 | Run 3 | Run 1 | Run 2 | Run 3 |
| | 2,560 | 3,410 | 2,000 | ND | 2,970 | ND |
| | 2,140 | 2,180 | ND | 1,880 | ND | 2,650 |
| | 1,780 | 1,810 | 2,210 | 2,030 | 630 | 1,590 |
| | 1,630 | 2,150 | 1,950 | 1,810 | 1,460 | ND |
| | 1,520 | 2,360 | 1,820 | 2,680 | 2,310 | 2,020 |
| | 1,960 | 2,390 | 1,860 | 1,760 | 1,600 | 2,060 |
| | 1,640 | 2,170 | 1,770 | 2,440 | 2,520 | 1,130 |
| | 2,060 | 1,740 | 1,800 | 1,780 | 2,240 | 1,700 |
| | 2,000 | 2,010 | 2,100 | 2,140 | 2,260 | 2,260 |
| | 2,130 | 1,750 | 2,240 | 2,340 | 1,740 | 1,900 |
| | 1,880 | 1,960 | 1,960 | 1,860 | 2,270 | 1,450 |
| | 1,830 | 3,030 | 2,150 | 2,490 | 1,280 | 2,180 |
| Average | 2021 | | | | | |
| Std Dev | 431 | | | | | |
| CV | 21% | | | | | |

### Example 3

The Internal Standard Can Correct for Capillary to Capillary Variation.

In this example we demonstrate that use of the invention is not limited to a particular type of electrophoresis or to cell lysates being used as the sample. The example differs from the previous examples in several important ways. Rather than examining ERK in a cell lysate, purified recombinant ERK2 is examined. The analyte is resolved from the peptide containing the same epitope by a size separation rather than an IEF. The sample is processed in a semiautomated way with the sample being introduced to one end of the capillary. Detection is again by chemiluminescence. The analysis used the average signal from the standards to normalize signal.

The sample was created by combining recombinant Erk2 (cat. no. 14-536, Upstate) and TAMRA labeled Erk peptide (example 1) in 1×SDS sample loading buffer (50 mM Tris-HCl at pH 8.8 and 1% SDS) at a final concentration of 0.23 mg/ml and 40.8 ng/ml respectively.

Five (5) cm sections of nano-volume capillaries were prepared as in U.S. Patent Application No. 60/833,060. Capillary action was used to (nearly) fill 12 capillaries with commercially available size-based separation polymer solution (Beckman PN 391-163). The remaining volume of the capillary (about 1 to 10 nl) was filled with the sample by capillary action to create 12 replicates. SDS-MW gel buffer was used as separation buffer. Separation was done at a constant voltage of 250V for 2000 seconds. Separated proteins were immobilized by irradiation with UV light for 60 seconds.

The sample, having been introduced by hand, could not be identical in all twelve replicates. However the quantitative standard of the invention can be used to correct for the differences in volume, removing most of the variation between the replicates.

After UV immobilization, capillaries were washed with TBST-CAB solution containing TBST (10 mM Tris-HCl at pH 7.5, 150 mM NaCl, 0.05% Tween20) and 5% CAB (Cetyltrimethyl ammonium bromide). Immobilized proteins were then incubated for 1 hr with anti-Erk1/2 antibodies (cat. no. 06-182, Upstate) diluted to 1:300 in TBST. This was followed by another TBST-CAB wash to remove non-specifically bound antibodies. Immobilized proteins were then incubated with HRP conjugated goat anti-rabbit secondary antibodies (cat. no. 81-6120, Zymed) diluted to 1:500 in TBST for 10 mins. Capillaries were washed with TBST-CAB and chemiluminescence detection was done used West Femto Stable Peroxide buffer and Luminol/Enhancer solution (cat. no. 1859023, 1859022, Pierce).

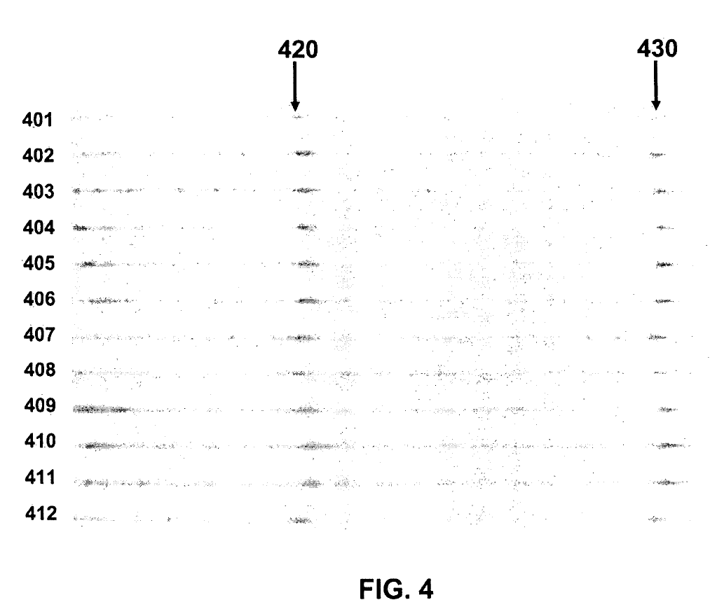

Images of the capillaries are taken using a CCD camera (Princeton Instruments) for 15 s, 30 s, 60 s, and 300 s. Images are stored in a 16 bit TIF file format and analyzed using Imagequant (Molecular Dynamics) and DAX (Van Mierlo Software Consultancy). FIG. 4 presents a TIFF image from a CCD camera of the 12 replicate samples of Example 3. These samples were separated by size in denaturing SDS PAGE. The image has been gamma corrected (1.98) for illustrative purposes, to bring out the background signal from the capillaries 401 through 412. The standard of the invention 420 and the analyte 430 are clearly visible above the background in all 12 replicates. Again, one can see that the signal of the standard and the analyte increase and decrease relative to each other. The peak produced by the standard (420) can be used to correct the signal in analytes (430). Thus, the invention greatly reduces variability between capillaries.

The data and their analysis are shown in Table 5 below. As expected, the ERK2 peaks areas varied as different volumes of sample were added to the capillary. This variation was reduced 4 fold by using the quantitation standards of the invention.

TABLE 5

| | ERK2 Peak | Std Peak | SignalSTDobs | Corrected |
|---|---|---|---|---|
| | Area | Area | SignalSTDave | ERK2 Signal |
| | 55624 | 48432 | 0.84 | 66303 |
| | 56213 | 50806 | 0.88 | 63874 |
| | 114790 | 90841 | 1.57 | 72950 |
| | 68365 | 56844 | 0.98 | 69431 |
| | 26531 | 22053 | 0.38 | 69453 |
| | 59204 | 60473 | 1.05 | 56519 |
| | 69562 | 68602 | 1.19 | 58538 |
| | 71073 | 64821 | 1.12 | 63299 |
| | 57878 | 56703 | 0.98 | 58927 |
| Average | 64360 | | | 64366 |
| Std Dev | 23113 | | | 5651 |
| CV | 36% | | | 9% |

### Example 4

The Internal Standard Can Normalize for Capillary to Capillary Variation.

Presented is an example where a protein that binds to antibodies in a manner different than the analyte is used as a quantitative standard. The experiment was performed as in Example 1 with the following exception. Protein G (Biovision, Inc.) was used in place of the synthetic ERK1 peptide. This protein does not contain an ERK epitope. Antibodies to the ERK proteins bind to the Protein G at the Fc region of the antibody.

Protein G was added to a 0.3 mg/ml HELA lysate at a final concentration of 15.6 pg/μl prior to loading onto the capillary.

As seen in the results of this experiment shown in Table 6 below, an antibody targeted against the analytes was able to bind to protein G. Although the kinetics of the antibody(s) binding to protein G are probably very different than the kinetics of binding to the analytes, the signal from Protein G can still be used to normalize from capillary to capillary because the amount of protein added to each capillary was the same.

TABLE 6

| Capillary | Protein G | | | SignalSTDobs | ERK1 | ERK2 |
|---|---|---|---|---|---|---|
| Number | Signal | ERK1obs | ERK2obs | SignalSTDave | Normalized | Normalized |
| 1 | 84 | 558 | 1,315.39 | 1.33 | 419 | 987 |
| 2 | 44 | 293 | 895.53 | 0.70 | 422 | 1287 |
| 4 | 55 | 363 | 882.43 | 0.88 | 414 | 1007 |
| 5 | 96 | 594 | 1,580.35 | 1.53 | 388 | 1033 |
| 6 | 49 | 315 | 1,005.38 | 0.78 | 405 | 1293 |
| 8 | 60 | 329 | 1,094.51 | 0.96 | 344 | 1141 |
| 9 | 67 | 555 | 1,481.73 | 1.07 | 518 | 1383 |
| 10 | 63 | 400 | 1,107.39 | 1.01 | 396 | 1098 |
| 12 | 47 | 367 | 1,147.00 | 0.75 | 489 | 1530 |
| Average | | 419 | 1168 | | 422 | 1196 |
| Std Dev | | 117 | 245 | | 53 | 188 |
| CV | | 28% | 21% | | 12% | 16% |

### Example 5

The Internal Standard Can Be Used to Determine the Amount of an Unknown. (IEF and Epitope-Type Standard)

This example illustrates several aspects of the invention. The most important of which is the use of the invention to generate a standard curve for the determination of the amount of an unknown.

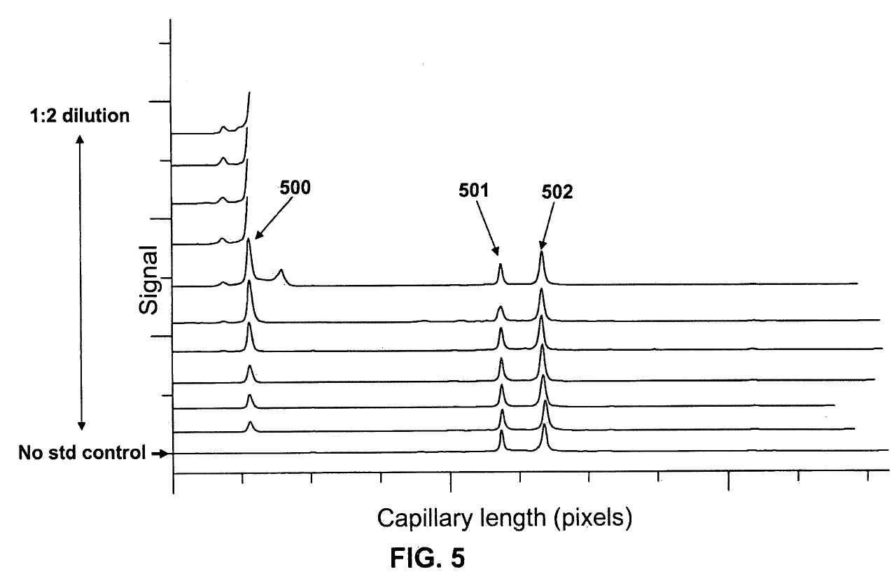

This experiment was performed as in Example 1 except that the amount of standard was titrated down in 2 fold dilutions from 0.5 nM to 1.0 pM concentration. The amount of lysate added remained constant—even in a ‘no standard’ control. Line traces extracted from the signal in the capillary in this experiment are shown in FIG. 5. Different amounts of a standard are added to a sample, separated, detected, and the data was extracted from the TIFF image. The top 10 lines of the graph show the results of the standard titration with the signal on the Y axis and the capillary length on the X. Reagents are flowed in from the left side. If there is too much signal the reagents become consumed and there is no signal to the right of the flowing reagents. At some dilution of the standard (500) the signal is no longer saturated and the data from entire length of the capillary is measurable including the analytes ERK1 (501) and ERK2 (502). The bottom line of the graph shows a lysate sample in which no standard was added.

In this particular example the signal from the standard (500) is so strong that it depletes the chemiluminescent reagents and no signal from the analytes, ERK1 and ERK2 are measurable. At some concentration (˜30 pm) the standard's signal is low enough to permit measurement of the analytes (501 and 501). All of the peak areas were measured and are presented in the Table 7 below.

TABLE 7

| | | |
|---|---|---|
| Standard | ERK1 | ERK |

| | [Peptide] | Peak | Peak | Peak |
|---|---|---|---|---|
| Capillary # | (pM) | Area | Area | Area |
| 1 | 0.0 | 0 | 148 | 251 |
| 2 | 1.0 | 80 | 147 | 287 |
| 3 | 2.0 | 106 | 160 | 282 |
| 4 | 3.9 | 132 | 162 | 302 |
| 5 | 7.8 | 222 | 153 | 288 |
| 6 | 16 | 362 | 136 | 275 |
| 7 | 31 | 535 | 141 | 287 |
| 8 | 63 | ND | ND | ND |
| 9 | 125 | ND | ND | ND |
| 10 | 250 | ND | ND | ND |
| 11 | 500 | ND | ND | ND |
| | | Ave | 150 | 282 |
| | | Dev | 9 | 16 |
| | | % CV | 6% | 6% |

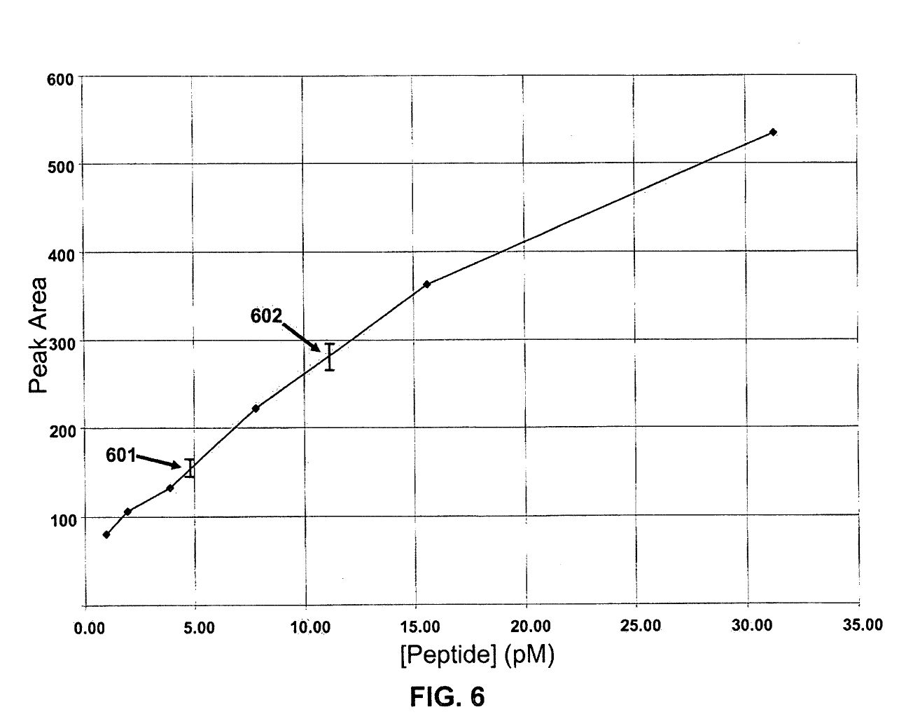

As expected, the signal of the standard increased with increasing amounts of the standard while the signal for the analytes remained fairly constant. This demonstrates that the use of a standard does not necessarily interfere with the measurement of the analyte. A standard curve was then generated by classical means; the concentration of the standard was plotted on the x axis and the signal generated was plotted on the Y. The results are shown in FIG. 6. The average signal of each analyte was then placed on the graph with error bars to indicate one standard deviation from the mean. ERK1 is indicated by 601 and ERK2 by 602. The peak areas of the standard can be plotted versus the concentration of the standard to create a standard curve. The line indicate the quantity of the amount of ERK1 (601) and ERK2 (602)

The concentration of ERK1 in the lysate generates the same signal as adding the internal standard to a concentration of 5 pM. With some extensive validation we may be able to prove that there is a one to one ratio between the signal from ERK1 and the signal from the standard but that isn't necessary. We could describe the concentration in terms of the standard and say the concentration of ERK1 is 5 peptide standard units (PSU). In other words, from day to day, instrument to instrument, one would expect that this sample will repeatedly be found to contain about 5 PSU of ERK1 and 11.5 PSU of ERK2.

### Example 6

The Internal Standard Can Be Used When Detecting by Fluorescence

This example demonstrates broader aspects of the invention. In this example the standard is a pure protein that shares an epitope with the analyte. The standard and the analyte are detected by scanning fluorescence rather than chemiluminescence. Although the analyte and the standard are themselves fluorescent, a dye-labeled antibody was used to enable a fluorescent immunoassay

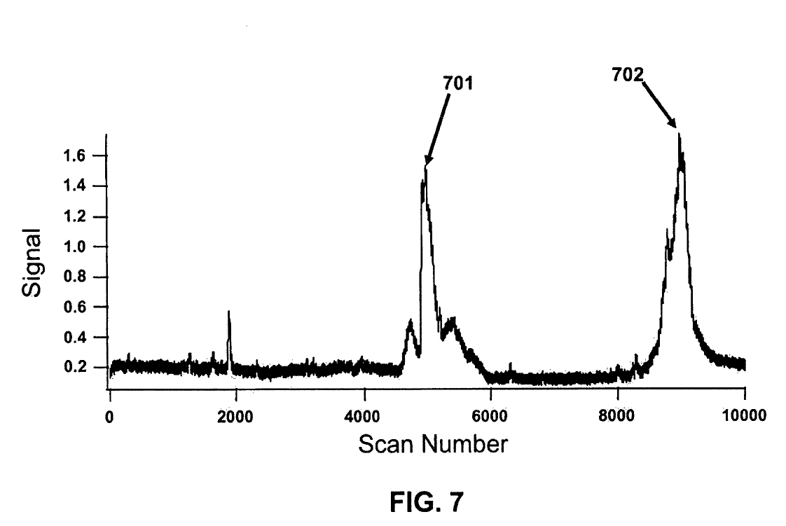

A cell lysate was prepared as described in Example 1 on cells that expressed an ERK2-GFP fusion protein. Recombinant green fluorescent protein (rGFP) was added to a final concentration of 5 μg/ml to the cell lysate. The mixture was diluted to three different amounts and assayed as described in Example 1 with the following exceptions. The sample was first probed with a 1 to 500 dilution of anti-GFP antibody (Invitrogen; cat# A1122). An Alexa647-labeled anti-rabbit antibody (Invitrogen, cat# A21244) was used at a 1 to 500 dilution as the secondary antibody. An instrument of in-house design Knittle J. E., et al., Laser Induced Fluorescence Detector for Capillary Based Iso-Electric Immunoblot Assay. 2007 (Manuscript in press, Analytical Chemistry), was used to detect the laser excited fluorescence. The Helium Neon laser was used to excite the Alexa647; emission was detected using a photomultiplier tube set at a variety of gain settings to demonstrate the ability of the quantitative standard of the invention to normalize the results. The results of a typical measurement from this experiment are shown in FIG. 7. The main rGFP peak 701 and the primary ERK-GFP fusion peaks 702 are labeled.

TABLE 8

| | Std Peak | Correction | ERK2-GFP | ERK2-GFP |
|---|---|---|---|---|
| Capillary | Height | Factor | signal | norm |
| 1 | 4.00 | 2.55 | 4.09 | 1.60 |
| 2 | 3.80 | 2.42 | 3.40 | 1.40 |
| 3 | 2.10 | 1.34 | 2.20 | 1.64 |
| 4 | 2.10 | 1.34 | 2.17 | 1.62 |
| 5 | 0.85 | 0.54 | 0.95 | 1.75 |
| 6 | 0.60 | 0.38 | 0.57 | 1.48 |
| 8 | 0.90 | 0.57 | 0.80 | 1.39 |
| 9 | 0.44 | 0.28 | 0.40 | 1.43 |
| 10 | 0.20 | 0.13 | 0.21 | 1.68 |
| 11 | 0.70 | 0.45 | 0.80 | 1.79 |
| | | Average | 1.56 | 1.58 |
| | | Std Dev | 1.34 | 0.15 |
| | | CV | 86% | 9% |

In this example the quantitative standard if recombinant green fluorescent protein, a well characterized substance that will cross react with the analyte, and ERK2-GFP fusion protein. The analyte is in a complex mixture, a cell lysate. Dilution and detector changes were introduced to artificially create variation in the results to be corrected by the standard. Use of the standard as taught was able to correct for the variation, in the analyte, from a CV of 86% to 9%. Thus, standards of the invention can be used to correct for variation due to sample manipulation and detector changes, making data that would be unusable, quite quantitative.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

All patents, patent applications, publications, and references cited herein are expressly incorporated by reference to the same extent as if each individual publication or patent application was specifically and individually indicated to be incorporated by reference.

We claim:

1. A method of measuring one or more analytes in a sample comprising:
adding a known quantity of a standard to said sample;
loading said sample into a microfluidic device;
separating said standard and said analyte by electrophoresis;
immobilizing said standard and said analyte;
detecting said standard and said analyte with at least one antibody; and
comparing a signal representative of said standard to a signal representative of said analyte.

2. The method according to claim 1 wherein said standard is detected with the same antibody as said analyte.

3. The method according to claim 1 wherein said electrophoresis is isoelectric focusing (IEF).

4. The method according to claim 3 wherein said IEF is performed in a capillary.

5. The method according to claim 1 wherein said electrophoresis separates the analyte by size.

6. The method according to claim 5 wherein said separation is performed in a capillary

7. The method according to claim 1 wherein said standard comprises an antigenic region linked to a mobility modifying region.

8. The method according to claim 1 wherein said standard comprises a peptide.

9. The method according to claim 8 wherein said peptide comprises all or part of the epitope used to generate the antibody.

10. The method according to claim 1 wherein said standard comprises a polypeptide that binds antibodies.

11. The method according to claim 10 wherein said standard is protein A or protein G.

12. The method according to claim 1 wherein said standard contains a whole protein.

13. The method according to claim 11 wherein said whole protein is a fusion protein.

14. The method according to claim 11 wherein said whole protein comprises all or part of the epitope used to generate the antibody.

15. A method of measuring at least one analyte in a sample, characterized in that: one or more analytes and a known quantity of standard are resolved and immobilized in a fluid path, and then detection reagents are conveyed through the fluid path which bind to or interact with the analytes and standard and permit detection and comparison of signals generated from said immobilized analytes and standard.

16. A kit for measuring at least one analyte in a sample, comprising:
one or more internal standards, and
one or more antibodies that detect the one or more standards.

17. The kit of claim 16 further comprising materials indicating the concentration of the one or more internal standards.

18. The kit of claim 16 wherein the one or more standards is comprised of a molecule that shares an epitope with an analyte of interest.

19. The kit of claim 16 further comprising: any one or more of capillaries, microfluidic devices, reactive moieties, buffers and detection agents.
